(19) United States
(12) Patent Application Publication
(10) Pub. No.: US 2023/0139065 A1
(43) Pub. Date: May 4, 2023

(54) Method and Apparatus for Enhancing Performance

(71) Applicant: S-Alpha Therapeutics, Inc., Seoul (KR)

(72) Inventors: Seung Eun Choi, Seoul (KR); Yong Han Kim, Suwon-si Gyeonggi-do (KR)

(73) Assignee: S-Alpha Therapeutics, Inc., Seoul (KR)

(21) Appl. No.: 18/075,844

(22) Filed: Dec. 6, 2022

Related U.S. Application Data
(63) Continuation-in-part of application No. PCT/KR2021/007299, filed on Jun. 10, 2021.
(60) Provisional application No. 63/037,203, filed on Jun. 10, 2020.

(51) Int. Cl. G16H 20/30; A61B 5/024; G16H 20/70; G16H 40/40; G16H 40/63; G16H 50/20; A61B 5/11

(52) U.S. Cl. CPC G16H 20/30; A61B 5/024; G16H 20/70; G16H 40/40; G16H 40/63; G16H 50/20; A61B 5/1118

(57) ABSTRACT

Methods and apparatuses are described herein for enhancing performance of a user. For example, an apparatus may generate one or more performance enhancing instructions that cause, based on the user's performance of the performance enhancing instructions, at least one physiological changes in the user's physiological mechanism to cope with at least one of anxiety or stress. The at least one physiological changes may include at least one of dopamine secretion induction, oxytocin secretion induction, cortisol level reduction, melatonin secretion induction, or synapsing promotion. The apparatus may then provide the user the one or more performance enhancing instructions via the apparatus. The one or more performance enhancing instructions may comprise a first/second concentration enhancing digital instruction, an anxiety/stress reducing digital instruction, and a coordination & agility enhancing digital instruction.

### CROSS-REFERENCE TO RELATED APPLICATION

This application claims under priority to and the benefit of U.S. Patent Application No. 63/037,203, filed Jun. 10, 2020, the disclosures of which are incorporated herein by reference in their entireties.

### TECHNICAL FIELD

### Background Art

Anxiety disorder is a type of mental disorder of which the feeling of anxiousness or fear for something that has not happened yet has been pathologically strong or long-lasting to disrupt one's daily life. A stress is a type of reaction that occurs to protect one's body when threatened or challenged by an outside factor, i.e., a stressor. It is difficult to eliminate the root cause of anxiety or stress itself in physiologically. Moreover, constant and excessive stress status may also disrupt one's daily life, developing into ‘Neurotic, stress-related and somatoform disorders. Accordingly, the feelings of anxiety or stress may significantly affect one's performance or ability to do something important in their life. For example, the feeling of anxiety decreases an athlete's performance ability, leading to unsatisfied results without showing the athlete's performance to the fullest.

Existing anxiety or stress coping methods are mostly involved in meditation, psychological relaxation, psychotherapy, game, mindfulness, stress management, etc. These methods are neither developed considering the user's neurohumoral factors and neuroplasticity, nor based on objective, scientific theory and evidence to cope with the anxiety and stress. The users rely heavily on subjective reviews of other reviewers rather than evidence-based evaluation. Thus, methods and apparatuses that cope with anxiety and/or stress based on neurohumoral factors and neuroplasticity thereby enabling evidence-based evaluation are needed.

### DISCLOSURE

### Technical Solution

Methods and apparatuses are described herein for enhancing performance. For example, an apparatus may generate one or more performance enhancing instructions that are designed to cause, based on the user's performance of the one or more performance enhancing instructions, at least one physiological changes in the user's physiological mechanism to cope with at least one of anxiety or stress. The apparatus may then provide the user the one or more performance enhancing instructions via the apparatus. The at least one physiological changes may include at least one of dopamine secretion induction, oxytocin secretion induction, cortisol level reduction, melatonin secretion induction, or synapsing promotion. The one or more performance enhancing instructions comprise a first concentration enhancing digital instruction related to the dopamine secretion induction, a second concentration enhancing digital instruction related to the melatonin secretion induction, an anxiety/stress reducing digital instruction related to at least one of the oxytocin secretion induction or the cortisol level reduction, and a coordination & agility enhancing digital instruction related to the synapsing promotion.

### DESCRIPTION OF DRAWINGS

A more detailed understanding may be had from the following description, given by way of example in conjunction with the accompanying drawings, wherein like reference numerals in the figures indicate like elements, and wherein:

FIG. 1 is a diagram illustrating an example mechanism of action (MOA) for enhancing performance;

FIG. 2 is a diagram illustrating an example anticipated neurohumoral status change after inducing positive neurotranmitters and/or synapsing;

FIG. 3 is a diagram illustrating an example maximization of the performance of an athlete;

FIG. 4 is a diagram illustrating an example physiological mechanism decreasing an athlete's performance due to anxiety/stress;

FIG. 5 is a diagram illustrating an example MOA of overcoming an athlete's anxiety through instructions;

FIG. 6 is a diagram illustrating an example patient assistance in the recovery from the operation of intraocular lens (IOL) implants/multifocal lens in ophthalmology;

FIG. 7 is a diagram illustrating an example physiological mechanism of forming anxiety that can develop in a patient's body undergoing ophthalmology operation before and/or after the surgery;

FIG. 8 is a diagram illustrating an example MOA of overcoming a patient's anxiety through behavioral language;

FIG. 9 is a diagram illustrating an example performance enhancing application (PEA) that copes with anxiety and stress of users;

FIG. 10 is a diagram illustrating an example of PEA;

FIG. 11 is a diagram illustrating examples of instruction input and practice monitoring of PEA; and

FIG. 12 is a system diagram illustrating an example device that can be used for PEA.

FIG. 13 is a diagram illustrating stressor disease diagnostic criteria for PEA and relationship with sensors.

FIG. 14 is a diagram illustrating a data sensing standardization for PEA.

FIG. 15 is a diagram illustrating physical part analysis using motion detection.

FIG. 16 is a diagram illustrating a process of setting goals for athletes using PEA.

### MODE FOR INVENTION

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

Diagnostic and Statistical Manual of Mental Disorders (DSM), published by the American Psychiatric Association, is most widely used in terms diagnosing mental disorders. According to the classification of DSM-V (5th edition), anxiety disorder is a type of mental disorder of which the feeling of anxiousness or fear for something that has not happened yet has been pathologically strong or long-lasting to disrupt one's daily life. It includes generalized anxiety disorder, a phobia (specific phobia, social phobia, agoraphobia), panic disorder, obsessive-compulsive disorder, post-traumatic stress disorder, and separation anxiety disorder. If left untreated, it can lead to depression and addiction, and can be a risk factor of dementia or suicide.

Even if not pathologically diagnosed as an anxiety disorder, an average person may feel particularly anxious in certain situations, and the uncontrolled feeling of anxiousness sometimes lasts in undesirable actions/results.

Currently, a number of anxiety (allegedly) reducing applications are available on Google App Store, most of which are developed involving meditation, psychological relaxation, psychotherapy, brain exercise, meta cognition, mind control, etc. These apps target unspecified individuals, and the users rely heavily on subjective reviews of other reviewers rather than evidence-based evaluation to use the apps.

A stress reaction is a type of reaction that occurs to protect one's body when threatened or challenged by an outside factor, i.e., a stressor. In a well-managed stress status, the feeling of tension causes stress hormones to awaken the brain and thereby to help improve memory. Also, in the incipient stages, cortisol, a type of stress hormone, enhances the body's immunity. However, the 10th revision of the International Statistical Classification of Diseases and Related Health Problems (ICD-10) has classified constant and excessive stress status into ‘Neurotic, stress-related and somatoform disorders (F40-F48)’.

Aside from these pathological stress statuses, for instance, an athlete may fail to show better performance in an important game due to excessive mental stress.

Currently, numerous anti-stress or stress (allegedly) reducing applications are available on Google App Store, most of which are developed involving meditation, relaxation, funny game, psychotherapy, mindfulness, stress management, etc. These apps target unspecified individuals, and the users rely heavily on subjective reviews of other reviewers rather than evidence-based evaluation to use the apps.

Neuroplasticity refers to the human brain's ability to change throughout one's life experiences. Results of recent researches demonstrate that learning or other environmental factors make neurons continuously develop or deteriorate. Particularly, the hippocampus, a part of the brain responsible for memory, enhances neuroplasticity significantly by incessantly developing new neurons and deteriorating the old neurons. The frontal area of brain is the location of active neuronal myelination, resulting in synapsing and neuroplasticity too. The BDNF protein is known to target the synapses of neurons especially to promote neuroplasticity.

In particular, myelin formation and remodeling of oligodendrocyte, which has been developed from oligodendrocyte precursor cells (OPC), plays a very important role in terms of learning, exercising, and making up for the impaired brain regions. Increasing neuroplasticity leads to neurons' synapsing and thereby contributes to the enhancement of coordination and agility.

At the status quo, there is no application which applies the notion of neuroplasticity with the enhancement of coordination and agility. There are only applied cases where brain training and brain game are used at a rudimentary level.

In order to overcome these limitations of the existing apps, 1) target users of the app need to be specified, 2) evidence-based evaluation needs to proceed through app development based on the hypothesis which has been through medical evidences and scientific ones.

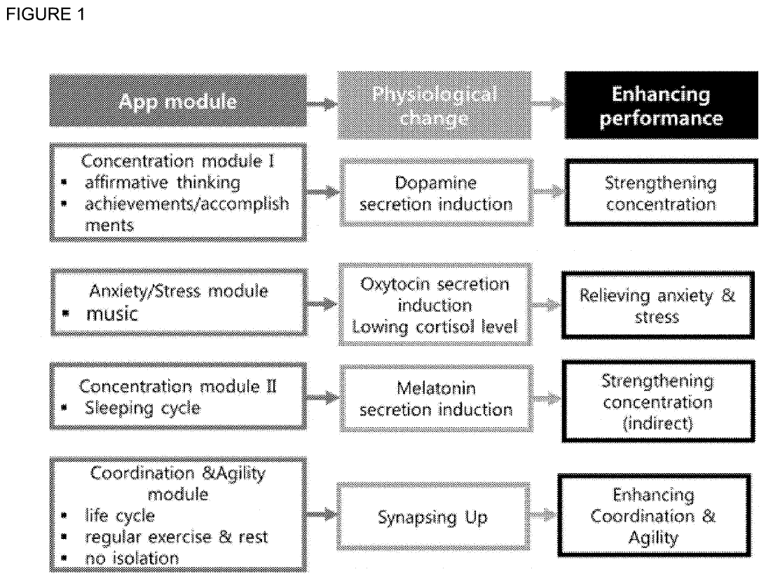

FIG. 1 illustrates an example mechanism of action (MOA) for enhancing performance, which may be used in combination with any of other embodiments described herein.

By practicing the instructions that have been delivered to the user via the application, neurohumoral factors and neuroplasticity, which cope with anxiety and stress in physiological terms, increase. This, in turn, improves the users concentration, overcome anxiety and pressure, and enhances coordination and agility. Consequently, the user's performance can be enhanced.

It is difficult to eliminate the root cause of anxiety or stress itself in physiologically, except for pathological anxiety disorder and neurological stress. For example, although an athlete may undergo significant anxiety and stress before athletics or competition, patient before a scheduled operation, an artist before a recital/show, and a student before an exam, feeling anxiety and stress is a part of the athletics/recovery/recital/show/exam/etc. in terms of physiological process.

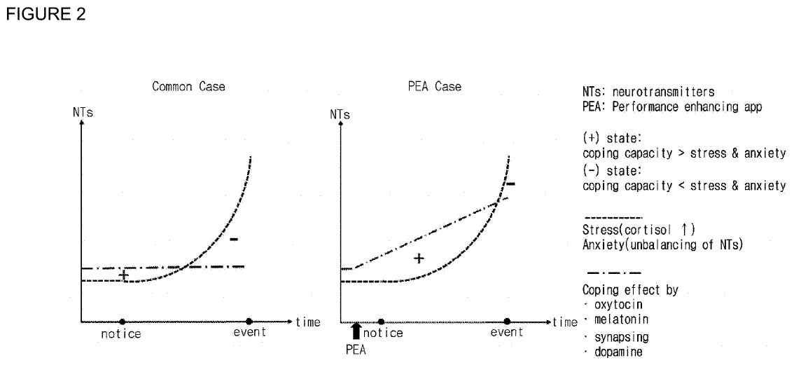

FIG. 2 illustrates an example anticipated neurohumoral status change after inducing positive neurotranmitters and/or synapsing, which may be used in combination with any of other embodiments described herein. As illustrated in FIG. 2, even under the condition where there are limits in eliminating the root cause of anxiety or stress, the negative impacts of anxiety or stress may be reduced by “coping with” or boosting up positive neurotransmitters (e.g., dopamine, oxytocin, and/or melatonin) or synapsing.

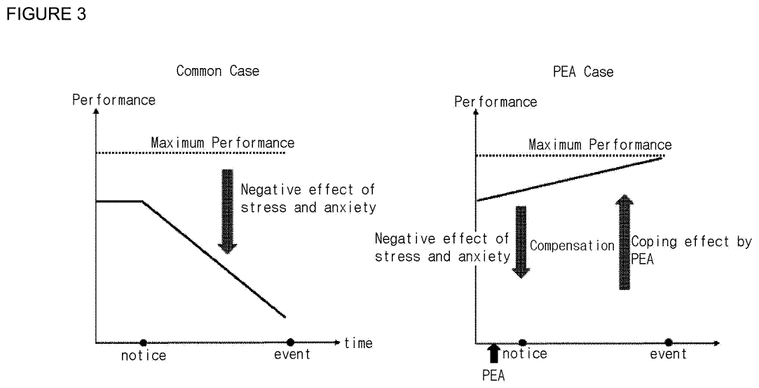

FIG. 3 illustrates an example maximization of the performance of an athlete, which may be used in combination with any of other embodiments described herein. Athletes may undergo significant anxiety prior to athletics or major competitions. Anxiety may be triggered as a result of the combination of possibilities where he might lose the tournament in athletics or make mistakes, concerns of potential criticism from audiences, and a sense of duty to win the game. The feeling of anxiety decreases the athlete's performance ability, leading to unsatisfiable results without showing the athlete's performance to the fullest.

By using the performance enhancing app (PEA), athletes may cope with anxiety and stress to enhance concentration, coordination, and agility, and reach his/her fullest potential.

The existing methods to overcome athletes' anxiety consists of relaxation training, thought stopping, labeling, and deep breathing along with sufficient sleeping, exercise, complex carbohydrates intake, caffeine/sugar intake limit, etc. The relaxation training includes yoga, meditation, breathing exercise, pilates, etc. Thought stopping includes changing negative thoughts into positive thoughts and task-centered approach rather than result-centered. Labeling includes letting one perceive his/her own thoughts and emotions.

However, the above method, fore example, to overcome sports performance anxiety, do not compensate for the changes in physiological neurohumoral factors of the body occurring before the athlete's big/important games. In other words, it is symptomatic therapy for anxiety.

Meanwhile, the physiological mechanism for an athlete to overcome anxiety such as sufficient sleeping, exercise, complex carbohydrates intake, caffeine/sugar intake limit and others has not yet been specifically identified.

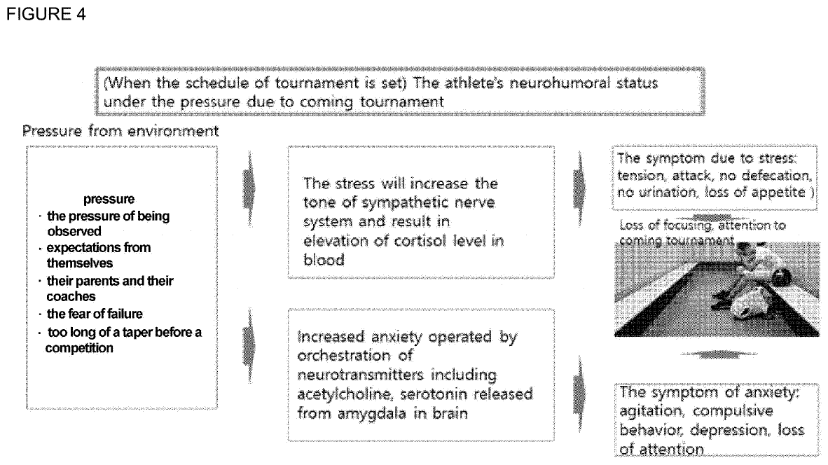

FIG. 4 illustrates an example physiological mechanism decreasing an athlete's performance due to anxiety/stress before competitions, which may be used in combination with any of other embodiments described herein. As illustrated in FIG. 4, the athletes' anxiety may be formed before games/athletics. This kind of anxiety formation may involve orchestration of neurotransmitters (NTs), and hence it may be difficult to identify a single, particular NT as the major source of the anxiety formation.

Among various NTs which cause anxiety, four NTs may be suggested as the neurohumoral factors which can result in coping over anxiety formation, i.e., increasing of dopamine, increasing of oxytocin, decreasing of cortisol, increasing of melatonin. In addition, synapsing regarding brain myelin formation and remodeling may also be suggested as the neuronal, cellular, and molecular mechanism of inducing coping-skill over anxiety formation.

The control using drugs of the present mechanism is not likely to be accounted as antagonizing mechanism, due to the fact that the relevant NTs with anxiety/stress formation are the most basic NTs to maintain the body's homeostasis in physiological terms. In other words, it may bring serious side-effect and safety issues, provided that it could be a target molecule to overcome anxiety. Even pushing aside these difficulties, the control of NT using drugs cannot be a solution to athletes' anxiety because it may trigger doping issues.

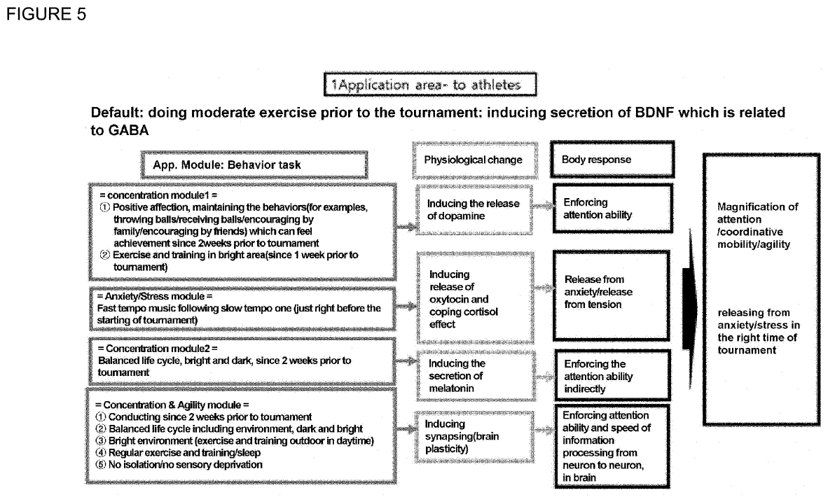

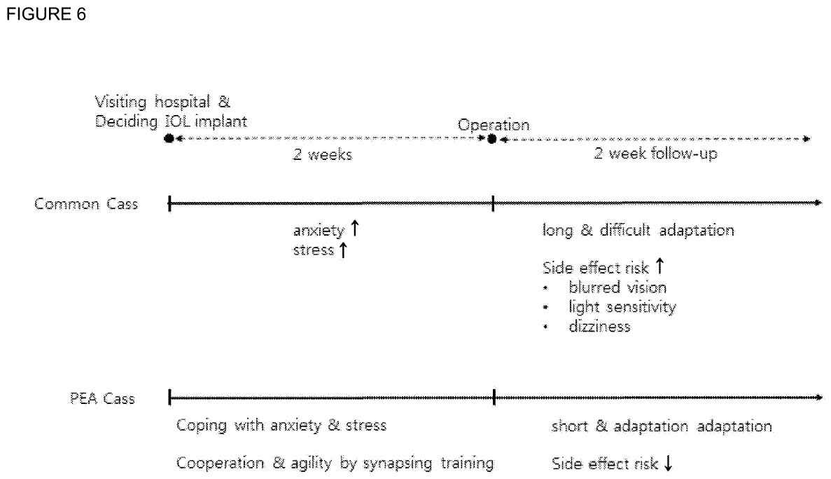

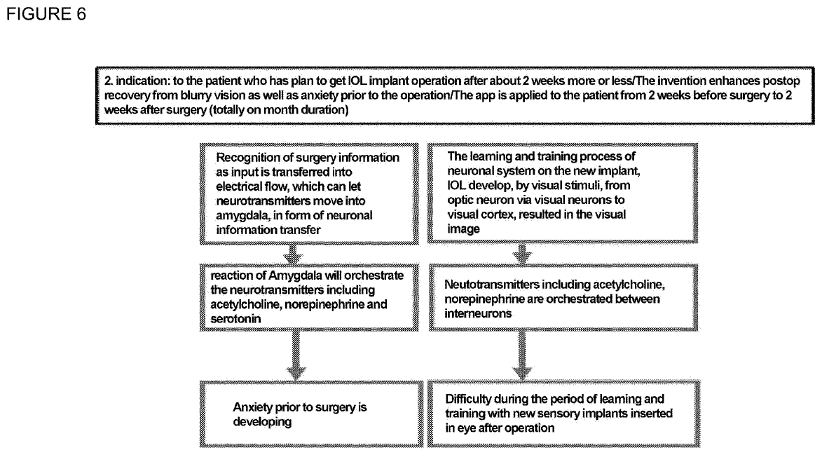

FIG. 5 illustrates an example MOA of overcoming an athlete's anxiety through instructions, which may be used in combination with any of other embodiments described herein. For example, an athlete's performance and ability may be maximized through instruction-practice which is delivered to the athlete via the application for two weeks prior to the game, based on the physiological mechanism of athletes' anxiety and stress formation. The MOA (mode of action or mechanism of action) to overcoming athletes' anxiety through the behavioral language may be illustrated as in FIG. 5. The MOA may comprise one or more modules (e.g., four modules). Specifically, concentration module 1 may include, but are not limited to: positive thinking, and executing instructions (e.g., for 2 weeks) that can make one feel a small sense of achievement constantly (e.g., throwing balls, cheer from family members, or cheer from friends.). Anxiety/Stress module may include, but are not limited to: listening up-tempo music after down-tempo music (e.g., before the start of the game). Concentration module 2 may include, but are not limited to, providing normal day and night environment (e.g., for 2 weeks). Coordination & Agility module may include, but are not limited to: starting the module at least 2 weeks earlier to the game, having proper life cycle, daylight (e.g., outdoor exercise during the day), regular exercise and sleeping, and no isolation/no sensory deprivation. The corresponding changes in NT and synapsing are summarized in FIG. 6 physiological changes column (the green box). These physiological changes are changed into physical changes (the black box in FIG. 6), and eventually, an athlete can relieve anxiety and pressure during the game and maximize his/her concentration, coordination, and agility.

In one aspect, the present disclosure relates to a method of enhancing performance of a user, the method comprising providing, by an electronic device to the user, one or more first modules selected from the group consisting of a concentration module I for inducing dopamine secretion, an anxiety and/or stress module, a concentration module II for inducing melatonin secretion, and a coordination and agility module, each of the one or more first modules comprising one or more first instructions for the user to follow. In some embodiments, the electronic device (i) comprises a sensor sensing adherence by the user to the first instructions of the one or more first modules, (ii) transmits adherence information based on the adherence, to a server, and (iii) receives one or more second instructions from the server based on the adherence information. In additional embodiments, the method further comprise providing, by the electronic device to the user, one or more second modules selected from the group consisting of a concentration module I for inducing dopamine secretion, an anxiety and/or stress module, a concentration module II for inducing melatonin secretion, and a coordination and agility module, the one or more second modules comprising the one or more second instructions.

In some embodiments, said one or more first modules comprise at least two, three or four of a concentration module I for inducing dopamine secretion, an anxiety and/or stress module, a concentration module II for inducing melatonin secretion, and a coordination and agility module. In some embodiments, said one or more first modules comprise all of a concentration module I for inducing dopamine secretion, an anxiety and/or stress module, a concentration module II for inducing melatonin secretion, and a coordination and agility module. In some embodiments, said one or more second modules comprise at least two, three or four of a concentration module I for inducing dopamine secretion, an anxiety and/or stress module, a concentration module II for inducing melatonin secretion, and a coordination and agility module. In some embodiments, said one or more second modules comprise all of a concentration module I for inducing dopamine secretion, an anxiety and/or stress module, a concentration module II for inducing melatonin secretion, and a coordination and agility module.

In some embodiments, the one or more first modules comprise the concentration module I to strengthen concentration of the user. In some embodiments, the concentration module I comprises one or more first instructions directed to affirmative thinking, achievement, and accomplishment. In additional embodiments, the one or more instructions comprise one or more instructions for positive affection, maintaining behaviors, exercise, and training in a bright area. In further embodiments, the concentration module I includes instructions to train in a bright area at least one week before a performance.

In some embodiments, the one or more first modules comprise the anxiety and/or stress module to relieve anxiety and stress. In some embodiments, the anxiety and/or stress module comprises one or more first instructions to induce oxytocin secretion and/or lower cortisol level. In additional embodiments, the one or more first instructions comprise one or more instructions to listen to music. In further embodiments, the electronic device receives and plays the sounds of the music. In yet further embodiments, the one or more first instructions comprise one or more instructions to listen to music in less than two or one hours before the performance.

In some embodiments, the one or more first modules comprise the concentration module II to strengthen concentration of the user. In some embodiments, and the concentration module II comprises one or more first instructions directed to sleeping cycle. In additional embodiments, the one or more instructions comprise one or more instructions for a balanced life cycle and brightness of the user's environment. In further embodiments, the one or more instructions comprise one or more instructions for sleeping cycle at least one or two weeks before the performance.

In some embodiments, the one or more first modules comprise the coordination and agility module to enhance coordination and agility. In some embodiments, and the coordination and agility module comprises one or more first instructions to promote synapsing. In additional embodiments, the one or more first instructions comprise one or more instructions for a balanced life cycle, a regular exercise and rest, brightness of the user's environment, and no isolation or sensory deprivation. In further embodiments, the coordination and agility module is provided to the user at least one or two weeks before a performance.

In some embodiments, the adherence is an adherence to the first instructions of the concentration module I, and the adherence information based on the adherence to the first instructions of the concentration module I is used to generate the second instructions only of the concentration module I. In some embodiments, the adherence is an adherence to the first instructions of the anxiety and/or stress module, and the adherence information based on the adherence to the first instructions of the anxiety and/or stress module is used to generate the second instructions only of the anxiety and/or stress module. In some embodiments, the adherence is an adherence to the first instructions of the concentration module II, and the adherence information based on the adherence to the first instructions of the concentration module II is used to generate the second instructions only of the concentration module II. In some embodiments, the adherence is an adherence to the first instructions of the a coordination and agility module, and the adherence information based on the adherence to the first instructions of the coordination and agility module is used to generate the second instructions only of the a coordination and agility module.

In some embodiments, the user is a musician, and the performance is musical performance. In some embodiments, the user is an athlete, and the performance is athletic performance. In some embodiments, the user is a baseball player. In some embodiments, the user is a skier.

In some embodiments, the user is a patient who underwent the operation of Intraocular lens/multifocal lens implant in ophthalmology. Embodiments for a patient who underwent the operation of Intraocular lens/multifocal lens implant in ophthalmology are described herein.

FIG. 6 illustrates an example patient assistance in the recovery from the operation of intraocular lens (IOL) implants/multifocal lens in ophthalmology, which may be used in combination with any of other embodiments described herein.

IOL implants surgery usually takes about 2 weeks of waiting period after getting admission approval notice for surgery in ophthalmology area, until the actual surgery takes place. During this period, some patients feel extremely anxious, which negatively influences on postop recovery and satisfaction of operation result. To add on, there is a need for auxiliary measures to help patients overcome the side-effects after the surgery and adjust to IOL.

The PEA described herein has positive impacts on enhancing postop recovery from blurry vision and satisfaction about surgery itself to maximizing neuroplasticity, inducing release of BDNF as well as to relieve patients' anxiety over the surgery through coping skills, by doing instructions practiced for 2 weeks before or after the surgery.

In the present disclosure, athletes and IOL implants inserted into patients are exemplified to describe the details of the invention, however, the present invention is not limited to the cases of athletes and IOL implants inserted patients. The target users may include, but are not limited to, artists, students, singers, contestees, and others who have to show performances after a given period of time.

For IOL implants surgery, an education program on wearing an eye patch for protecting the affected part after the surgery, applying eyewash, eye exercises, eye health and others has been conducted before the surgery depending on each hospital's condition. However, as the education program takes place right before the surgery, it is reported to be not as effective, worsening the patients' anxiety.

Therefore, there is a need for education programs after the surgery at an appropriate time which can reduce patients' anxiety for the surgery and at the same time helps the effective management of the patients. However, such endeavors to reduce anxiety before and after the eye surgery have merely been made so far.

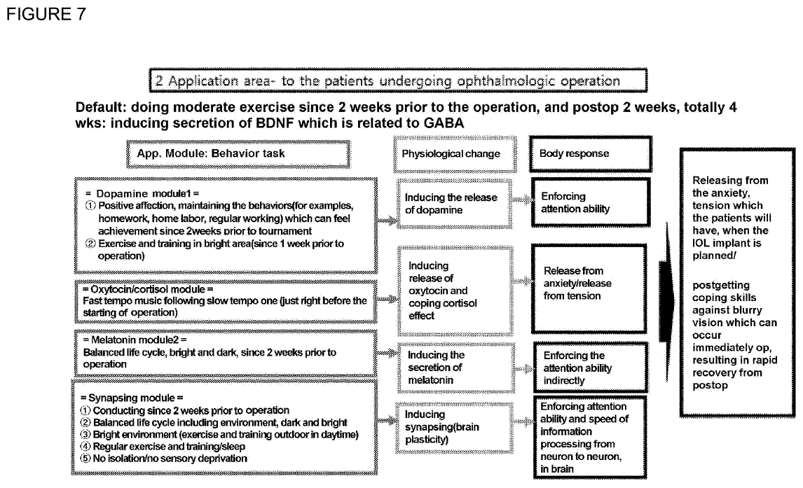

FIG. 7 illustrates an example physiological mechanism of forming anxiety that can develop in a patient's body undergoing ophthalmology operation before and/or after the surgery, which may be used in combination with any of other embodiments described herein. As illustrated in FIG. 7, patient's anxiety may be formed before, during and/or after the surgery. The formation of anxiety may be affected by the orchestration of various NTs, and thus it is difficult to define a single, particular NT as the mechanism of the anxiety formation.

The physiological mechanism of resulting in coping with an athlete's anxiety formation and that of anxiety formed by a patient who will undergo ophthalmology surgery with local anesthesia are the same or substantially similar. The four NTs—increasing of dopamine secretion, increasing of oxytocin secretion, decreasing or suppression of the effect of cortisol, increasing of melatonin secretion—used in physiological mechanism which can induce coping skill over athletes' anxiety/stress formation may be suggested as the neurohumoral factors of coping over anxiety/stress formation. In addition, the synapsing related to brain myelin formation and remodeling may be suggested as the cellular and molecular mechanism which can induce coping skill over anxiety/stress formation.

The relevance of each factor which can induce coping skill over anxiety/stress formation is described above as in the physiological mechanism which can induce coping skill over athletes' anxiety/stress formation.

The control of the physiological mechanism can induce coping skill over anxiety/stress formation through behavioral language. The control using drugs of the present mechanism is not likely to be accounted as antagonistic due to the fact that the relevant NTs are the most basic NTs to maintain the body's homeostasis in physiological terms. Anxiety/stress coping involving the application, unlike drug prescriptions, can prevent side-effect and safety issues.

In an embodiment, it may take up to four weeks (e.g., (two weeks each, before and after the surgery) for a patient who had IOL surgery to overcome the patients' anxiety through instruction-practice which is delivered to the patient via the application. Once the operation schedule is set for treatment, and pre-operation or post-operation care is needed before or after surgery using the application described herein. The application may be based on the physiological mechanism of patients' anxiety/stress formation.

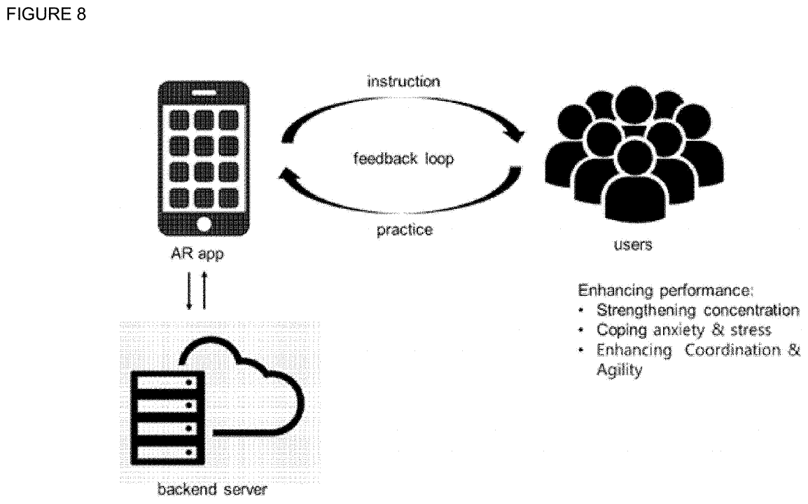

FIG. 8 illustrates an example MOA of overcoming a patient's anxiety through behavioral language, which may be used in combination with any of other embodiments described herein. The MOA may comprise one or more modules (e.g., four). For example, dopamine module may include, but are not limited to, positive thinking, executing instructions (for 2 weeks) that can make one feel a small sense of achievement constantly (doing chores, continuing with the job, etc.), and maintaining the life style in the light (starting from a week before the surgery). Oxytocin/Cortisol module may include, but are not limited to, listening up-tempo music after down-tempo music (e.g., a few hours earlier to the surgery). Melatonin module may include, but are not limited to, providing normal day and night environment (e.g., 2 weeks before the surgery). Synapsing module may include, but are not limited to, starting the module at least 2 weeks before the surgery, having proper life cycle, daylight (e.g., outdoor exercise during the day), regular exercise and sleeping, and no isolation/no sensory deprivation. The corresponding changes in NT and synapsing are summarized in FIG. 8 physiological changes column (the green box). These physiological changes are changed into physical changes (the black box in FIG. 8), and eventually, a patient can relieve anxiety before and after the surgery and minimize the side-effects of the surgery.

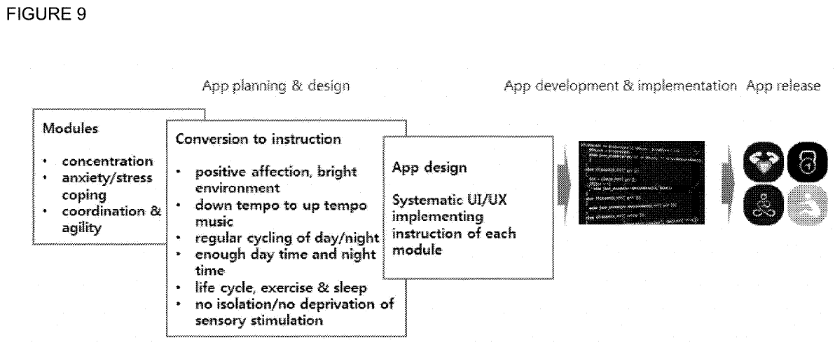

FIG. 9 illustrates an example performance enhancing application (PEA) that copes with anxiety and stress of a user, which may be used in combination with any of other embodiments described herein. The process of which the PEA reduces the users' anxiety is illustrated in FIG. 9. The PEA may be embodied or implemented in the form of application in digital devices such as a desktop computer, a laptop computer, a tablet, a smartphone, an IoT device, a wireless transmit/receive unit (WTRU) or the like. A user may download the application, receive instructions via PEA UI, and practice them. The user's practice may give feedback to the application through various means, for example, passive data gathering using log-in/out information, user review/evaluation, sensors, etc. The instruction-practice feedback loop may increase the users' adherence to induce their constant and voluntary participation. Taking a closer look at the specific physiological responses by the PEA, the following may be induced: secretion of dopamine, oxytocin and melatonin, suppression of cortisol, and neurons' synapsing as a result of the instruction-practice process. The PEA may be designed based on the MOA that induces the aforementioned physiological responses. Consequently, a user such as an athlete or a patient can actively participate in overcoming anxiety or stress through the practiced instructions to maximize performance abilities with enhanced concentration, coordination and agility.

In some embodiments, the server disclosed herein receives the one or more second instructions from an external reviewer. In additional embodiments, the external reviewer comprises a health professional. In additional embodiments, the external reviewer comprises an artificial intelligence (AI).

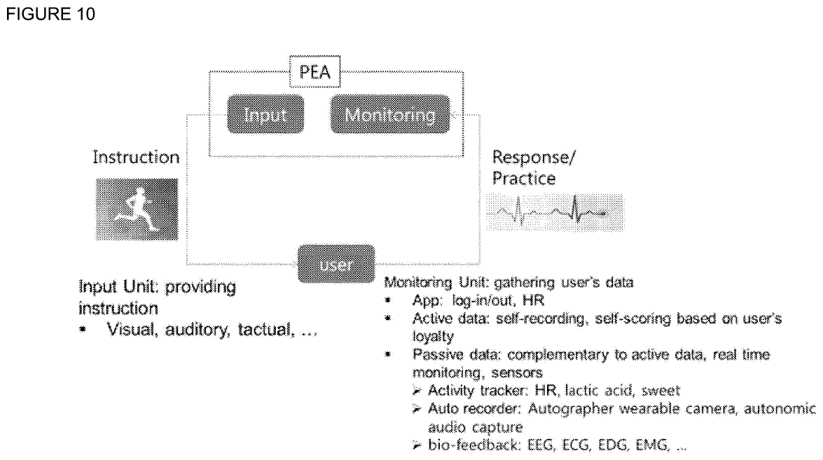

FIG. 10 illustrates an example of PEA, which may be used in combination with any of other embodiments described herein. PTA may specify the instructions of the concentration, anxiety/stress coping, coordination & agility modules as described in FIGS. 5 and 8, and reflect them in the application design. The PEA can be made and be launched based on the application programming that reflects the aforementioned application planning.

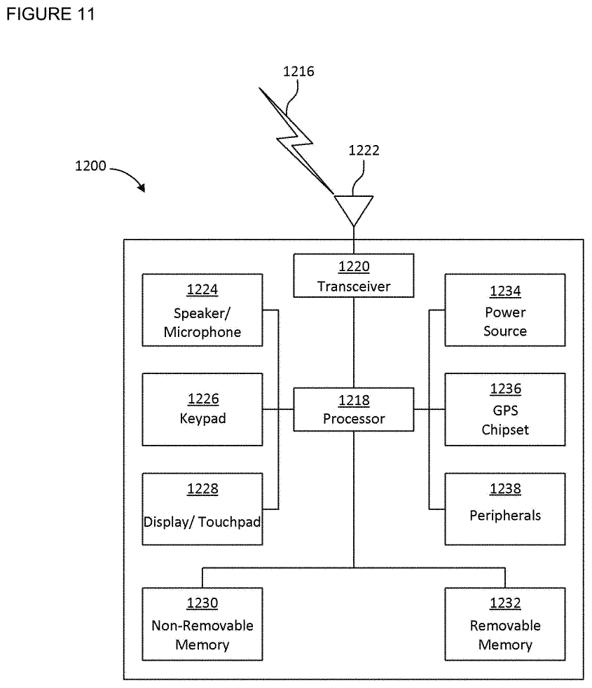

FIG. 11 illustrates examples of instruction input and practice monitoring of PEA, which may be used in combination with any of other embodiments described herein. The instruction input may be provided to a user by visual display, auditory narration, touch/vibration, etc. The practice out can be classified to three categories: 1) application log-in/out information, 2) active data which are generated by user's typing or recording, 3) passive data which are gathered by sensors. The sensors for passive data gathering include activity trackers, auto recorders, bio-feedback instruments.

In some embodiments, the sensor described herein comprises one or more of: a motion sensor, a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer. In additional embodiments, the sensor comprises the GPS sensor and the heart rate sensor. In further embodiments, the sensor comprises a touch sensor, and the user provides the adherence information to the electronic device using the touch sensor.

In some embodiments, the sensor described herein detects positions of knees and/or ankles of the user, and/or the distances thereof. In further embodiments, the sensor described herein detects positions of the neck, shoulder, arms and/or hip of the user, and/or the distances thereof. In further embodiments, the sensor described herein detects positions of the fingers of the user, and/or the distances thereof.

In some embodiments, the sensor captures motions of the athletic performance. In additional embodiments, the sensor captures at least 5, 10, 15, 20, 25, or 30 images per second.

In another aspect, the present disclosure is directed to a system for enhancing performance of a user, comprising: a digital apparatus configured to execute a digital application comprising one or more first modules described herein, for enhancing performance of a user, wherein the digital apparatus comprises a sensor described above for sensing adherence by the user to a first set of instructions of the one or more first modules. In some embodiments, the system also comprise a healthcare provider portal configured to provide one or more options to a healthcare provider to perform one or more tasks to prescribe performance enhancement of a user based on information received from the digital application. In some embodiments, the system also comprise an administrative portal configured to provide one or more options to an administrator of the system to perform one or more tasks to manage access to the system by the healthcare provider. In additional embodiments, the digital application instructs a processor of the digital apparatus to execute operations comprising: generating digital therapeutic modules for enhancing performance based on a mechanism of action in and a therapeutic hypothesis for the enhancing performance of a user. In further embodiments, the generating of the digital therapeutic modules comprises generating the digital therapeutic modules based on biochemical factors related to enhance performance of a user.

In some embodiments, the one or more options provided to the healthcare provider are selected from the group consisting of adding or removing the user, viewing or editing personal information for the user, viewing adherence information for the user, viewing a result of the user for one or more at least partially completed digital therapeutic modules, prescribing one or more digital therapeutic modules to the user, altering a prescription for one or more digital therapeutic modules, and communicating with the user.

In some embodiments, the one or more options comprise the viewing or editing personal information for the user, and the personal information comprises one or more selected from the group consisting of an identification number for the user, a name of the user, a date of birth of the user, an email of the user, an email of the guardian of the user, a contact phone number for the user, a prescription for the user, and one or more notes made by the healthcare provider about the user.

In some embodiments, the personal information comprises the prescription for the user, and the prescription for the user comprises one or more selected from the group consisting of a prescription identification number, a prescription type, a start date, a duration, a completion date, a number of scheduled or prescribed digital therapeutic modules to be performed by the user, and a number of scheduled or prescribed digital therapeutic modules to be performed by the user per day.

The system of any one of embodiments 43-70, wherein the one or more options comprise the viewing the adherence information, and the adherence information of the user comprises one or more of a number of scheduled or prescribed digital therapeutic modules completed by the user, and a calendar identifying one or more days on which the user completed, partially completed, or did not complete one or more scheduled or prescribed digital therapeutic modules.

In some embodiments, the one or more options comprise the viewing the result of the user, and the result of the user for one or more at least partially completed digital therapeutic modules comprises one or more selected from the group consisting of a time at which the user started a scheduled or prescribed digital therapeutic module, a time at which the user ended a scheduled or prescribed digital therapeutic module, an indicator of whether the scheduled or prescribed digital therapeutic module was fully or partially completed, and an exercise intensity (EI).

In some embodiments, the one or more options provided to the administrator of the system are selected from the group consisting of adding or removing the healthcare provider, viewing or editing personal information for the healthcare provider, viewing or editing de-identified information of the user, viewing adherence information for the user, viewing a result of the user for one or more at least partially completed digital therapeutic modules, and communicating with the healthcare provider.

In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.

In some embodiments, the one or more options comprise the viewing or editing the de-identified information of the user, and the de-identified information of the user comprises one or more selected from the group consisting of an identification number for the user, and the healthcare provider for the user.

In some embodiments, the one or more options comprise the viewing the adherence information for the user, and the adherence information of the user comprises one or more of a number of scheduled or prescribed digital therapeutic modules completed by the user, and a calendar identifying one or more days on which the user completed, partially completed, or did not complete one or more scheduled or prescribed digital therapeutic modules.

In some embodiments, the one or more options comprise the viewing the result of the user, and the result of the user for one or more at least partially completed digital therapeutic modules comprises one or more selected from the group consisting of a time at which the user started a scheduled or prescribed digital therapeutic module, a time at which the user ended a scheduled or prescribed digital therapeutic module, an indicator of whether the scheduled or prescribed digital therapeutic module was fully or partially completed, and an exercise intensity (EI).

In some embodiments, the digital application further comprises a push alarm and/or push notifications for reminding the subject to complete a digital therapeutic module.

In some embodiments, the digital apparatus comprises: a digital instruction generation unit configured to generate digital therapeutic modules for enhancing performance of the user, generate digital instructions based on the digital therapeutic modules, and provide the digital instructions to the user; and an outcome collection unit configured to collect the user's execution outcomes of the digital instructions. In additional embodiments, the digital instruction generation unit generates the digital therapeutic modules based on the inputs from the healthcare provider. In additional embodiments, the digital instruction generation unit generates the digital therapeutic modules based on information received from the user.

In some embodiments, the information is received from the user comprises at least one of basal factors, medical information, and digital therapeutics literacy of the user, the basal factors including the user's activity, heart rate, sleep, and diet, the medical information including the user's electronic medical record (EMR), family history, genetic vulnerability, and genetic susceptibility, and the digital therapeutics literacy including the user's accessibility, and technology adoption to the digital therapeutics and the apparatus.

In some embodiments, the outcome collection unit collects the execution outcomes of the digital instructions by monitoring the user's adherence to the digital instructions or allowing the user to directly input the user's adherence to the digital instructions.

In some embodiments, the generation of the digital instructions at the digital instruction generation unit and the collection of the user's execution outcomes of the digital instructions at the outcome collection unit are repeatedly executed several times with multiple feedback loops. In additional embodiments, the digital instruction generation unit generates the user's digital instructions for this cycle based on the user's digital instructions in the previous cycle and the execution outcome data on the user's digital instructions in the previous cycle collected at the outcome collection unit.

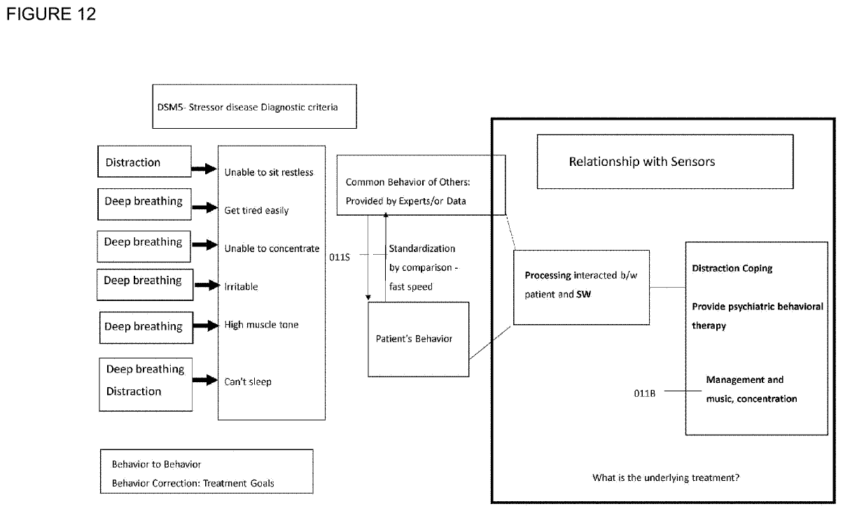

FIG. 12 illustrates an example device 1200 that can be used for PEA, which may be used in combination with any of other embodiments described herein. As shown in FIG. 12, the device 1200 may include a processor 1218, a transceiver 1220, a transmit/receive element 1222, a speaker/microphone 1224, a keypad 1226, a display/touchpad 1228, non-removable memory 1230, removable memory 1232, a power source 1234, a global positioning system (GPS) chipset 1236, and/or other peripherals 1238, among others. It will be appreciated that the device 1200 may include any sub-combination of the foregoing elements while remaining consistent with an embodiment. By way of example, the device 1200 may include a mobile device, a user equipment (UE), a mobile station, a fixed or mobile subscriber unit, a subscription-based unit, a pager, a cellular telephone, a personal digital assistant (PDA), a smartphone, a laptop, a netbook, a personal computer, a wireless sensor, a hotspot or Mi-Fi device, an Internet of Things (IoT) device, a watch or other wearable, a head-mounted display (HMD), a vehicle, a drone, a medical device and applications (e.g., remote surgery), an industrial device and applications (e.g., a robot and/or other wireless devices operating in an industrial and/or an automated processing chain contexts), a consumer electronics device, a device operating on commercial and/or industrial wireless networks, and the like.

The processor 1218 may be a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field

Programmable Gate Arrays (FPGAs), any other type of integrated circuit (IC), a state machine, and the like. The processor 1218 may perform data processing, power control, input/output processing, sensor date processing, and/or any other functionality that enables the device 1200 to provide antiviral digital vaccine. The processor 1218 may be coupled to the transceiver 1220, which may be coupled to the transmit/receive element 1222. While FIG. 12 depicts the processor 1218 and the transceiver 1220 as separate components, it will be appreciated that the processor 1218 and the transceiver 1220 may be integrated together in an electronic package or chip.

The transmit/receive element 1222 may be configured to transmit data to, or receive data from a sever located in a medical institution or institution that manages the PEA. For example, instructions from a doctor or a coach and medical information sensed from a user may be received/transmitted from/to the server, via a base station over the air interface 1216. In one embodiment, the transmit/receive element 1222 may be an antenna configured to transmit and/or receive RF signals. In an embodiment, the transmit/receive element 1222 may be an emitter/detector configured to transmit and/or receive IR, UV, or visible light signals, for example. In yet another embodiment, the transmit/receive element 1222 may be configured to transmit and/or receive both RF and light signals. It will be appreciated that the transmit/receive element 1222 may be configured to transmit and/or receive any combination of wireless signals. The transceiver 1220 may be configured to modulate the signals that are to be transmitted by the transmit/receive element 1222 and to demodulate the signals that are received by the transmit/receive element 1222.

The processor 1218 of the device 1200 may be coupled to, and may receive user input data from, the speaker/microphone 1224, the keypad 1226, the display/touchpad 1228 (e.g., a liquid crystal display (LCD) display unit or organic light-emitting diode (OLED) display unit) and/or the peripherals 1238 (e.g., sensors or digital camera). The processor 1218 may also output user data or digital instructions to the speaker/microphone 1224, the keypad 1226, the display/touchpad 1228 and/or the peripherals 1238. In addition, the processor 1218 may access information from, and store data in, any type of suitable memory, such as the non-removable memory 1230 and/or the removable memory 1232. The non-removable memory 1230 may include random-access memory (RAM), read-only memory (ROM), a hard disk, or any other type of memory storage device. The removable memory 1232 may include a subscriber identity module (SIM) card, a memory stick, a secure digital (SD) memory card, and the like. In other embodiments, the processor 218 may access information from, and store data in, memory that is not physically located on the device 1200, such as on a server or a home computer (not shown).

The processor 1218 may receive power from the power source 1234, and may be configured to distribute and/or control the power to the other components in the device 1200. The power source 1234 may be any suitable device for powering the device 1200. For example, the power source 1234 may include one or more dry cell batteries (e.g., nickel-cadmium (NiCd), nickel-zinc (NiZn), nickel metal hydride (NiMH), lithium-ion (Li-ion), etc.), solar cells, fuel cells, and the like.

The processor 1218 may also be coupled to the GPS chipset 1236, which may be configured to provide location information (e.g., longitude and latitude) regarding the current location of the device 1200. In addition to, or in lieu of, the information from the GPS chipset 1236, the device 1200 may receive location information over the air interface 1216 from a base station and/or determine its location based on the timing of the signals being received from two or more nearby base stations. It will be appreciated that the device 1200 may acquire location information by way of any suitable location-determination method while remaining consistent with an embodiment.

The processor 1218 may further be coupled to other peripherals 1238, which may include one or more software and/or hardware modules that provide additional features, functionality and/or wired or wireless connectivity. For example, the peripherals 1238 may include an accelerometer, an e-compass, a satellite transceiver, a digital camera (for photographs and/or video), a universal serial bus (USB) port, a vibration device, a television transceiver, a hands free headset, a Bluetooth® module, a frequency modulated (FM) radio unit, a digital music player, a media player, a video game player module, an Internet browser, a Virtual Reality and/or Augmented Reality (VR/AR) device, an activity tracker, and the like. The peripherals 1238 may include one or more sensors. The sensors may be one or more of a gyroscope, an accelerometer, a hall effect sensor, a magnetometer, an orientation sensor, a proximity sensor, a temperature sensor, a time sensor; a geolocation sensor, an altimeter, a light sensor, a touch sensor, a magnetometer, a barometer, a gesture sensor, a biometric sensor, a humidity sensor and the like.

In another aspect, the present disclosure relates to a computing system for enhancing performance of a user described herein, comprising: a display configured to provide, to the user, one or more first modules selected from the group consisting of a concentration module I for inducing dopamine secretion, an anxiety and/or stress module, a concentration module II for inducing melatonin secretion, and a coordination and agility module, each of the one or more first modules comprising one or more first instructions for the user to follow as disclosed above; a sensor described herein configured to sense adherence by the user to the instructions of the one or more first modules; a transmitter configured to transmit adherence information based on the adherence, to a server; and a receiver configured to receive, from the server, one or more second instructions based on the adherence information. In some embodiments, the display is further configured to provide, to the user, one or more second modules selected from the group consisting of a concentration module I for inducing dopamine secretion, an anxiety and/or stress module, a concentration module II for inducing melatonin secretion, and a coordination and agility module, each of the one or more second modules comprising the one or more second instructions.

In some embodiments, the digital application for enhancing performance instructs a processor of the digital apparatus to execute operations comprising: generating digital therapeutic modules for enhancing performance based on a mechanism of action in and a therapeutic hypothesis for enhancing performance. In some embodiments, the generating of the digital therapeutic modules comprises generating the digital therapeutic modules based on biochemical factors related to the performance enhancement.

In another aspect, the present disclosure relates to a non-transitory computer readable medium having stored thereon software instructions for enhancing performance of the user described above that, when executed by a processor, cause the processor to: display, by an electronic device to the user, the one or more first modules described above, each of the one or more first modules comprising instructions for the user to follow; sense, by the sensor described above in the electronic device, adherence by the user to the instructions of the one or more first modules; transmit, by the electronic device, adherence information based on the adherence, to a server; receive, from the server, one or more second instructions based on the adherence information; and display, to the user, the one or more second modules described above, the one or more second modules comprising the one or more second instructions.

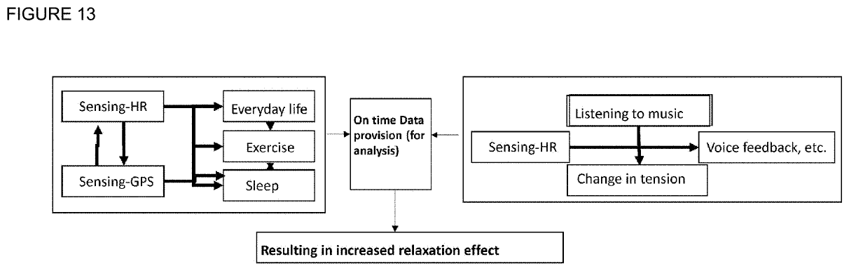

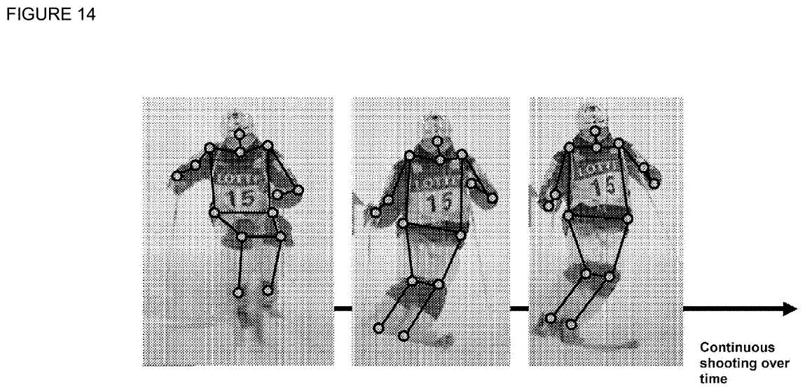

FIG. 13 is a diagram illustrating exemplary stressor disease diagnostic criteria for PEA and relationship with sensors, and FIG. 14 is a diagram illustrating a data sensing standardization for PEA. It is impossible for humans to provide on-time prevention and treatment functions by connecting sensors and sensors (requires fast information processing function). Thus, an exemplary embodiment of the present disclosure enables adding the device technique to the sensing and preventing/treating patients. Referring to FIG. 13, in 011S, a motion capture can be used to collect individual movement data and standardize the movement of ski players in real time (a technique of standardizing 30 times per second by deep camera, for example), by standardizing to increase analysis clarity and showing results different from other related techniques. The data may be analyzed so that the standard playing input into the device or the playing can be corrected retrospectively by a coach, for example. It may be a device that can perform behavior correction on time, or by sending the behavior correction feedback of the athlete in real time. In some embodiments, the device may have a calibration function based on the real-time data from the athlete and/or external data, such as data from coach, already built-in data, and control data. The device may also provide feedback to an athlete by continuously calibrating the athlete's motions and analyzing the motions in real time compared to the past performances. In 011b, data can be processed through HR sensor data and a SW algorithm interlocking GPS (how quickly and accurately quantify recognition), and the processed data can be provided to the designated personals (e.g., skiers) on time, as illustrated in FIG. 14. In some embodiments, 011b is the end point of the whole system, that is, the part that ultimately manages the mental (relieving anxiety and tension, and giving the effect of concentration) in the device. Here, it may be a device to help instantly concentrate and relax in real time when the heart rate does not stabilize by using voice or other methods to stabilize the heart rate (e.g. deep breathing, listening to music, or thinking about something good) while sensing the heart rate more precisely in real time.

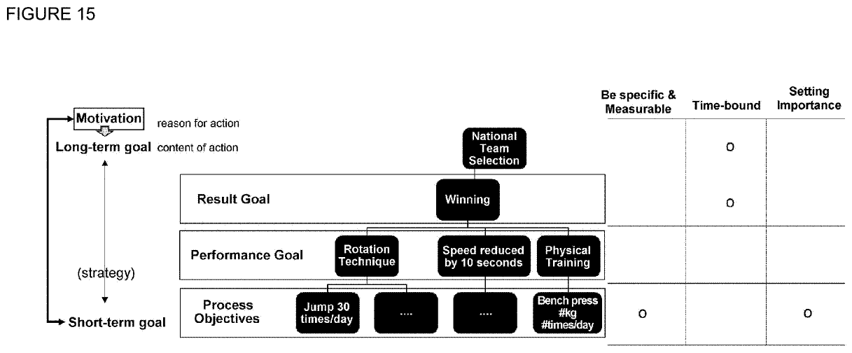

FIG. 15 is a diagram illustrating an exemplary physical part analysis using motion detection. For example, each body part can be analyzed, such as close stance of both knees and ankles, movement of the pelvis, an upper body lower back, neck shake, wrist movement, etc., to analyze the posture of the skier using motion detection.

In further embodiments, when the user is a skier, the sensor described herein detects positions of knees and/or ankles of the skier, and the distances thereof. In further embodiments, the sensor described herein detects positions of the neck, shoulder, arms and/or hip of the skier.

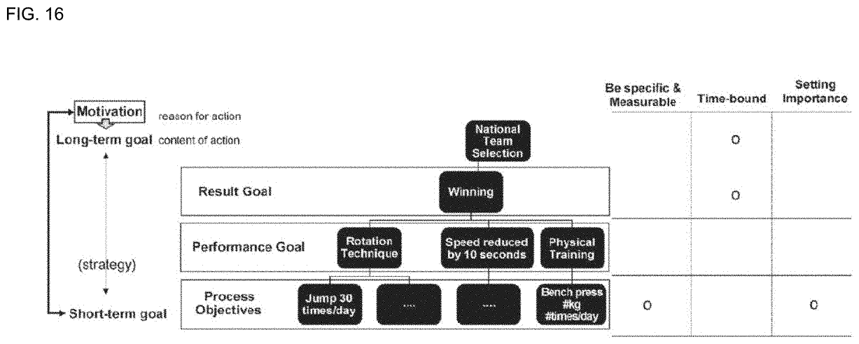

FIG. 16 is a diagram illustrating an exemplary process of setting goals for athletes using PEA. A goal setting is important in planning how players think, feel and behave, and it is important to set effective and correct goals. According to the present disclosure, the application may provide a way to provide information when setting goals without user-input, and it is possible to modify the goal setting in the event of an injury, slump, failure, etc. The application may further provide necessity and method and may include information such as evaluation frequency, evaluation contents (why the goal is achieved or not achieved, records of assessments and advice from leaders, etc.). Such a goal setting can be the driving force to achieve the set goals. The user (e.g., skiers) can check whenever motivation is necessary.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs). A processor in association with software may be used to implement a radio frequency transceiver for use in a WTRU, UE, terminal, base station, RNC, or any host computer.

In another aspect, the present disclosure is related to the following embodiments.

Embodiment 1. A method for use in an apparatus to enhance performance of a user, the method comprising:

generating one or more performance enhancing instructions that cause, based on the user's performance of the one or more performance enhancing instructions, at least one physiological changes in the user's physiological mechanism to cope with at least one of anxiety or stress; and

providing the user the one or more performance enhancing instructions via the apparatus.

Embodiment 2. The method of embodiment 1, wherein the at least one physiological changes includes at least one of dopamine secretion induction, oxytocin secretion induction, cortisol level reduction, melatonin secretion induction, or synapsing promotion.

Embodiment 3. The method of embodiment 1 or 2, wherein the one or more performance enhancing instructions comprise a first concentration enhancing digital instruction, a second concentration enhancing digital instruction, an anxiety/stress reducing digital instruction, and a coordination & agility enhancing digital instruction.

Embodiment 4. The method of embodiment 3, wherein the first concentration enhancing digital instruction comprises affirmative thinking, achievement, and accomplishment that are designed to cause the dopamine secretion induction in the user's physiological mechanism.

Embodiment 5. The method of embodiment 3, wherein the second concentration enhancing digital instruction comprises a sleeping cycle that is designed to cause the melatonin secretion induction in the user's physiological mechanism.

Embodiment 6. The method of embodiment 3, wherein the anxiety/stress reducing digital instruction comprises music that is designed to cause at least one of the oxytocin secretion induction or the cortisol level reduction in the user's physiological mechanism.

Embodiment 7. The method of embodiment 3, wherein the coordination & agility enhancing digital instruction comprises a life cycle, a regular exercise and rest, and no isolation that are designed to cause the synapsing promotion in the user's physiological mechanism.

Embodiment 8. The method of embodiment 1, wherein the user is an athlete or a patient who had an operation of Intraocular lens/multifocal lens (IOL) implant in ophthalmology.

Embodiment 9. An apparatus for enhancing performance of a user, the apparatus comprising:

a processor configured to:

generate one or more performance enhancing instructions that cause, based on the user's performance of the one or more performance enhancing instructions, at least one physiological changes in the user's physiological mechanism to cope with at least one of anxiety or stress; and provide the user the one or more performance enhancing instructions.

Embodiment 10. The apparatus of embodiment 9, wherein the at least one physiological changes includes at least one of dopamine secretion induction, oxytocin secretion induction, cortisol level reduction, melatonin secretion induction, or synapsing promotion.

Embodiment 11. The apparatus of embodiment 10, wherein the one or more performance enhancing instructions comprise a first concentration enhancing digital instruction, a second concentration enhancing digital instruction, an anxiety/stress reducing digital instruction, and a coordination & agility enhancing digital instruction.

Embodiment 12. The apparatus of embodiment 11, wherein the first concentration enhancing digital instruction comprises affirmative thinking, achievement, and accomplishment that are designed to cause the dopamine secretion induction in the user's physiological mechanism, and wherein the second concentration enhancing digital instruction comprises a sleeping cycle that is designed to cause the melatonin secretion induction in the user's physiological mechanism.

Embodiment 13. The apparatus of embodiment 11, wherein the anxiety/stress reducing digital instruction comprises music that is designed to cause at least one of the oxytocin secretion induction or the cortisol level reduction in the user's physiological mechanism.

Embodiment 14. The apparatus of embodiment 11, wherein the coordination & agility enhancing digital instruction comprises a life cycle, a regular exercise and rest, and no isolation that are designed to cause the synapsing promotion in the user's physiological mechanism.

Embodiment 15. The apparatus of embodiment 11, wherein the user is an athlete or a patient who had an operation of Intraocular lens/multifocal lens (IOL) implant in ophthalmology.

Embodiment 16. A method for enhancing performance of an athlete, the method comprising:

performing, by the athlete, one or more performance enhancing instructions that causes at least one physiological changes in the athlete's physiological mechanism to cope with at least one of anxiety or stress.

Embodiment 17. The method of embodiment 16, wherein the at least one physiological changes includes at least one of dopamine secretion induction, oxytocin secretion induction, cortisol level reduction, melatonin secretion induction, or synapsing promotion.

Embodiment 18. The method of embodiment 16, wherein the one or more performance enhancing instructions comprise a first concentration enhancing digital instruction, a second concentration enhancing digital instruction, an anxiety/stress reducing digital instruction, and a coordination & agility enhancing digital instruction.

Embodiment 19. A method for enhancing performance of a patient, the method comprising:

performing, by the patient, one or more performance enhancing instructions that causes at least one physiological changes in the athlete's physiological mechanism to cope with at least one of anxiety or stress, wherein the at least one physiological changes includes at least one of dopamine secretion induction, oxytocin secretion induction, cortisol level reduction, melatonin secretion induction, or synapsing promotion.

Embodiment 20. The method of embodiment 19, wherein the one or more performance enhancing instructions comprise a first concentration enhancing digital instruction, a second concentration enhancing digital instruction, an anxiety/stress reducing digital instruction, and a coordination & agility enhancing digital instruction.

Embodiment 21. A method of enhancing performance of a user, the method comprising

providing, by an electronic device to the user, one or more first modules selected from the group consisting of a concentration module I for inducing dopamine secretion, an anxiety and/or stress module, a concentration module II for inducing melatonin secretion, and a coordination and agility module, each of the one or more first modules comprising one or more first instructions for the user to follow,

wherein the electronic device (i) comprises a sensor sensing adherence by the user to the first instructions of the one or more first modules, (ii) transmits adherence information based on the adherence, to a server, and (iii) receives one or more second instructions from the server based on the adherence information; and

providing, by the electronic device to the user, one or more second modules selected from the group consisting of a concentration module I for inducing dopamine secretion, an anxiety and/or stress module, a concentration module II for inducing melatonin secretion, and a coordination and agility module, the one or more second modules comprising the one or more second instructions.

Embodiment 22. The method of embodiment 21, wherein the one or more first modules comprise the

concentration module I to strengthen concentration of the user, and the concentration module I comprises one or more first instructions directed to affirmative thinking, achievement, and accomplishment.

Embodiment 23. The method of embodiment 22, wherein the one or more instructions comprise one or more instructions for positive affection, maintaining behaviors, exercise, and training in bright area.

Embodiment 24. The method of any one of embodiments 21-23, wherein the one or more first modules comprise the anxiety and/or stress module to relieve anxiety and stress, and the anxiety and/or stress module comprises one or more first instructions to induce oxytocin secretion and/or lower cortisol level.

Embodiment 25. The method of embodiment 24, wherein the one or more first instructions comprise one or more instructions to listen to music.

Embodiment 26. The method of embodiment 25, wherein the electronic device receives and plays sounds for the music.

Embodiment 27. The method of any one of embodiments 21-26, wherein the one or more first modules comprise the concentration module II to strengthen concentration of the user, and the concentration module II comprises one or more first instructions directed to sleeping cycle.

Embodiment 28. The method of embodiment 27, wherein the one or more instructions comprise one or more instructions for a balanced life cycle and brightness of the user's environment.

Embodiment 29. The method of any one of embodiments 21-28, wherein the one or more first modules comprise the coordination and agility module to enhance coordination and agility, and the coordination and agility module comprises one or more first instructions to promote synapsing.

Embodiment 30. The method of embodiment 29, wherein the one or more first instructions comprise one or more instructions for a balanced life cycle, a regular exercise and rest, brightness of the user's environment, and no isolation or sensory deprivation.

Embodiment 31. The method of any one of embodiments 21-30, wherein the server receives the one or more second instructions from an external reviewer.

Embodiment 32. The method of embodiment 31, wherein the external reviewer comprises a health professional.

Embodiment 33. The method of embodiment 31, wherein the external reviewer comprises an artificial intelligence (AI).

Embodiment 34. The method of any one of embodiments 21-33, wherein the sensor comprises one or more of: a motion sensor, a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

Embodiment 35. The method of any one of embodiments 21-34, wherein the sensor comprises the GPS sensor and the heart rate sensor.

Embodiment 36. The method of any one of embodiments 21-35, wherein the sensor comprises a touch sensor, and the user provides the adherence information to the electronic device using the touch sensor.

Embodiment 37. The method of any one of embodiments 21-36, wherein the user is a musician, and the performance is musical performance.

Embodiment 38. The method of any one of embodiments 21-36, wherein the user is an athlete, and the performance is athletic performance.

Embodiment 39. The method of any one of embodiments 21-36, wherein the user is a baseball player.

Embodiment 40. The method of any one of embodiments 21-36, wherein the user is a skier.

Embodiment 41. The method of any one of embodiments 38-40, wherein the sensor captures motions of the athletic performance.

Embodiment 42. The method of embodiment 41, wherein the sensor captures at least 30 images per second.

Embodiment 43. A system for enhancing performance of a user, comprising:

a digital apparatus configured to execute a digital application comprising one or more first modules, for enhancing performance of a user, wherein the digital apparatus comprises a sensor for sensing adherence by the user to a first set of instructions of the one or more first modules;

a healthcare provider portal configured to provide one or more options to a healthcare provider to perform one or more tasks to prescribe performance enhancement of a user based on information received from the digital application; and an administrative portal configured to provide one or more options to an administrator of the system to perform one or more tasks to manage access to the system by the healthcare provider.

Embodiment 44. The system of embodiment 43, wherein the digital application instructs a processor of the digital apparatus to execute operations comprising:

generating digital therapeutic modules for enhancing performance based on a mechanism of action in and a therapeutic hypothesis for the enhancing performance of a user.

Embodiment 45. The system of embodiment 44, wherein the generating of the digital therapeutic modules comprises generating the digital therapeutic modules based on biochemical factors related to enhance performance of a user.

Embodiment 46. The system of any one of embodiments 43-45, wherein the one or more first modules are selected from the group consisting of a concentration module I for inducing dopamine secretion, an anxiety and/or stress module, a concentration module II for inducing melatonin secretion, and a coordination and agility module.

Embodiment 47. The system of any one of embodiments 43-46, wherein the one or more first modules comprise the concentration module I to strengthen concentration of the user, and the concentration module I comprises one or more first instructions directed to affirmative thinking, achievement, and accomplishment.

Embodiment 48. The system of embodiment 47, wherein the one or more instructions comprise one or more instructions for positive affection, maintaining behaviors, exercise, and training in bright area.

Embodiment 49. The system of any one of embodiments 46-48, wherein the one or more first modules comprise the anxiety and/or stress module to relieve anxiety and stress, and the anxiety and/or stress module comprises one or more first instructions to induce oxytocin secretion and/or lower cortisol level.

Embodiment 50. The system of embodiment 49, wherein the one or more first instructions comprise one or more instructions to listen to music.

Embodiment 51. The system of embodiment 50, wherein the electronic device receives and plays sounds for the music .

*174Embodiment 52. The system of any one of embodiments 46-51, wherein the one or more first modules comprise the concentration module II to strengthen concentration of the user, and the concentration module II comprises one or more first instructions directed to sleeping cycle.

Embodiment 53. The system of embodiment 52, wherein the one or more instructions comprise one or more instructions for a balanced life cycle and brightness of the user's environment.

Embodiment 54. The system of any one of embodiments 46-53, wherein the one or more first modules comprise the coordination and agility module to enhance coordination and agility, and the coordination and agility module comprises one or more first instructions to promote synapsing.

Embodiment 55. The system of embodiment 54, wherein the one or more first instructions comprise one or more instructions for a balanced life cycle, a regular exercise and rest, brightness of the user's environment, and no isolation or sensory deprivation.

Embodiment 56. The system of any one of embodiments 43-55, wherein the server receives the one or more second instructions from an external reviewer.

Embodiment 57. The system of any one of embodiments 43-56, wherein the external reviewer comprises a health professional.

Embodiment 58. The system of any one of embodiments 43-57, wherein the external reviewer comprises an artificial intelligence (AI).

Embodiment 59. The system of any one of embodiments 43-58, wherein the sensor comprises one or more of: a motion sensor, a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

Embodiment 60. The system of any one of embodiments 43-59, wherein the sensor comprises the GPS sensor and the heart rate sensor.

Embodiment 61. The system of any one of embodiments 43-60, wherein the sensor comprises a touch sensor, and the user provides the adherence information to the electronic device using the touch sensor.

Embodiment 62. The system of any one of embodiments 43-61, wherein the user is a musician, and the performance is musical performance.

Embodiment 63. The system of any one of embodiments 43-61, wherein the user is an athlete, and the performance is athletic performance.

Embodiment 64. The system of any one of embodiments 43-61, wherein the user is a baseball player.

Embodiment 65. The system of any one of embodiments 43-61, wherein the user is a skier.

Embodiment 66. The system of any one of embodiments 63-65, wherein the sensor captures motions of the athletic performance.

Embodiment 67. The system of embodiment 66, wherein the sensor captures at least 30 images per second.

Embodiment 68. The system of any one of embodiments 43-67, wherein the one or more options provided to the healthcare provider are selected from the group consisting of adding or removing the user, viewing or editing personal information for the user, viewing adherence information for the user, viewing a result of the user for one or more at least partially completed digital therapeutic modules, prescribing one or more digital therapeutic modules to the user, altering a prescription for one or more digital therapeutic modules, and communicating with the user.

Embodiment 69. The system of embodiment 68, wherein the one or more options comprise the viewing or editing personal information for the user, and the personal information comprises one or more selected from the group consisting of an identification number for the user, a name of the user, a date of birth of the user, an email of the user, an email of the guardian of the user, a contact phone number for the user, a prescription for the user, and one or more notes made by the healthcare provider about the user.

Embodiment 70. The system of embodiment 69, wherein the personal information comprises the prescription for the user, and the prescription for the user comprises one or more selected from the group consisting of a prescription identification number, a prescription type, a start date, a duration, a completion date, a number of scheduled or prescribed digital therapeutic modules to be performed by the user, and a number of scheduled or prescribed digital therapeutic modules to be performed by the user per day.

Embodiment 71. The system of any one of embodiments 43-70, wherein the one or more options comprise the viewing the adherence information, and the adherence information of the user comprises one or more of a number of scheduled or prescribed digital therapeutic modules completed by the user, and a calendar identifying one or more days on which the user completed, partially completed, or did not complete one or more scheduled or prescribed digital therapeutic modules.

Embodiment 72. The system of any one of embodiments 43-71, wherein the one or more options comprise the viewing the result of the user, and the result of the user for one or more at least partially completed digital therapeutic modules comprises one or more selected from the group consisting of a time at which the user started a scheduled or prescribed digital therapeutic module, a time at which the user ended a scheduled or prescribed digital therapeutic module, an indicator of whether the scheduled or prescribed digital therapeutic module was fully or partially completed, and an exercise intensity (EI).

Embodiment 73. The system of any one of embodiments 43-72, wherein the one or more options provided to the administrator of the system are selected from the group consisting of adding or removing the healthcare provider, viewing or editing personal information for the healthcare provider, viewing or editing de-identified information of the user, viewing adherence information for the user, viewing a result of the user for one or more at least partially completed digital therapeutic modules, and communicating with the healthcare provider.

Embodiment 74. The system of embodiment 73, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.

Embodiment 75. The system of embodiment 73 or 74, wherein the one or more options comprise the viewing or editing the de-identified information of the user, and the de-identified information of the user comprises one or more selected from the group consisting of an identification number for the user, and the healthcare provider for the user.

Embodiment 76. The system of any one of embodiments 43-75, wherein the one or more options comprise the viewing the adherence information for the user, and the adherence information of the user comprises one or more of a number of scheduled or prescribed digital therapeutic modules completed by the user, and a calendar identifying one or more days on which the user completed, partially completed, or did not complete one or more scheduled or prescribed digital therapeutic modules.

Embodiment 77. The system of any one of embodiments 43-76, wherein the one or more options comprise the viewing the result of the user, and the result of the user for one or more at least partially completed digital therapeutic modules comprises one or more selected from the group consisting of a time at which the user started a scheduled or prescribed digital therapeutic module, a time at which the user ended a scheduled or prescribed digital therapeutic module, an indicator of whether the scheduled or prescribed digital therapeutic module was fully or partially completed, and an exercise intensity (EI).

Embodiment 78. The system of any one of embodiments 43-77, wherein the digital application further comprises a push alarm and/or push notifications for reminding the subject to complete a digital therapeutic module.

Embodiment 79. The system of any one of embodiments 43-78, wherein the digital apparatus comprises:
a digital instruction generation unit configured to generate digital therapeutic modules for enhancing performance of the user, generate digital instructions based on the digital therapeutic modules, and provide the digital instructions to the user; and
an outcome collection unit configured to collect the user's execution outcomes of the digital instructions.

Embodiment 80. The system of any one of embodiment 79, wherein the digital instruction generation unit generates the digital therapeutic modules based on the inputs from the healthcare provider.

Embodiment 81. The system of any one of embodiments 79-80, wherein the digital instruction generation unit generates the digital therapeutic modules based on information received from the user.

Embodiment 82. The system of embodiment 82, wherein the information is received from the user comprises at least one of basal factors, medical information, and digital therapeutics literacy of the user,
the basal factors including the user's activity, heart rate, sleep, and diet,
the medical information including the user's electronic medical record (EMR), family history, genetic vulnerability, and genetic susceptibility, and
the digital therapeutics literacy including the user's accessibility, and technology adoption to the digital therapeutics and the apparatus.

Embodiment 83. The system of any one of embodiments 79-82, wherein the outcome collection unit collects the execution outcomes of the digital instructions by monitoring the user's adherence to the digital instructions or allowing the user to directly input the users adherence to the digital instructions.

Embodiment 84. The system of any one of embodiments 79-83, wherein the generation of the digital instructions at the digital instruction generation unit and the collection of the user's execution outcomes of the digital instructions at the outcome collection unit are repeatedly executed several times with multiple feedback loops, and
the digital instruction generation unit generates the user's digital instructions for this cycle based on the users digital instructions in the previous cycle and the execution outcome data on the user's digital instructions in the previous cycle collected at the outcome collection unit.

Embodiment 85. A computing system for enhancing performance of a user, comprising:

a display configured to provide, to the user, one or more first modules selected from the group consisting of a concentration module I for inducing dopamine secretion, an anxiety and/or stress module, a concentration module II for inducing melatonin secretion, and a coordination and agility module, each of the one or more first modules comprising one or more first instructions for the user to follow;

a sensor configured to sense adherence by the user to the instructions of the one or more first modules;

a transmitter configured to transmit adherence information based on the adherence, to a server; and

a receiver configured to receive, from the server, one or more second instructions based on the adherence information,

wherein the display is further configured to provide, to the user, one or more second modules selected from the group consisting of a concentration module I for inducing dopamine secretion, an anxiety and/or stress module, a concentration module II for inducing melatonin secretion, and a coordination and agility module, each of the one or more second modules comprising the one or more second instructions.

Embodiment 86. The computing system of embodiment 85, wherein the digital application for enhancing performance instructs a processor of the digital apparatus to execute operations comprising:

generating digital therapeutic modules for enhancing performance based on a mechanism of action in and a therapeutic hypothesis for enhancing performance.

Embodiment 87. The computing system of embodiment 86, wherein the generating of the digital therapeutic modules comprises generating the digital therapeutic modules based on biochemical factors related to the performance enhancement.

Embodiment 88. The computing system of any one of embodiments 85-87, wherein the one or more first modules are selected from the group consisting of a concentration module I for inducing dopamine secretion, an anxiety and/or stress module, a concentration module II for inducing melatonin secretion, and a coordination and agility module.

Embodiment 89. The computing system of any one of embodiments 85-88, wherein the one or more first modules comprise the concentration module I to strengthen concentration of the user, and the concentration module I comprises one or more first instructions directed to affirmative thinking, achievement, and accomplishment.

Embodiment 90. The computing system of embodiment 89, wherein the one or more instructions comprise one or more instructions for positive affection, maintaining behaviors, exercise, and training in bright area.

Embodiment 91. The computing system of any one of embodiments 85-90, wherein the one or more first modules comprise the anxiety and/or stress module to relieve anxiety and stress, and the anxiety and/or stress module comprises one or more first instructions to induce oxytocin secretion and/or lower cortisol level.

Embodiment 92. The computing system of embodiment 91, wherein the one or more first instructions comprise one or more instructions to listen to music.

Embodiment 93. The computing system of embodiment 92, wherein the electronic device receives and plays sounds for the music.

Embodiment 94. The computing system of any one of embodiments 85-93, wherein the one or more first modules comprise the concentration module II to strengthen concentration of the user, and the concentration module II comprises one or more first instructions directed to sleeping cycle.

Embodiment 95. The computing system of embodiment 94, wherein the one or more instructions comprise one or more instructions for a balanced life cycle and brightness of the user's environment.

Embodiment 96. The computing system of any one of embodiments 85-95, wherein the one or more first modules comprise the coordination and agility module to enhance coordination and agility, and the coordination and agility module comprises one or more first instructions to promote synapsing.

Embodiment 97. The computing system of embodiment 96, wherein the one or more first instructions comprise one or more instructions for a balanced life cycle, a regular exercise and rest, brightness of the user's environment, and no isolation or sensory deprivation.

Embodiment 98. The computing system of any one of embodiments 85-97, wherein the server receives the one or more second instructions from an external reviewer.

Embodiment 99. The computing system of embodiment 98, wherein the external reviewer comprises a health professional.

Embodiment 100. The computing system of embodiment 98, wherein the external reviewer comprises an artificial intelligence (AI).

Embodiment 101. The computing system of any one of embodiments 85-100, wherein the sensor comprises one or more of: a motion sensor, a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

Embodiment 102. The computing system of any one of embodiments 85-101, wherein the sensor comprises the GPS sensor and the heart rate sensor.

Embodiment 103. The computing system of any one of embodiments 85-102, wherein the sensor comprises a touch sensor, and the user provides the adherence information to the electronic device using the touch sensor.

Embodiment 104. The computing system of any one of embodiments 85-103, wherein the user is a musician, and the performance is musical performance.

Embodiment 105. The computing system of any one of embodiments 85-103, wherein the user is an athlete, and the performance is athletic performance.

Embodiment 106. The computing system of any one of embodiments 85-103, wherein the user is a baseball player.

Embodiment 107. The computing system of any one of embodiments 85-105, wherein the user is a skier.

Embodiment 108. The computing system of any one of embodiments 105-107, wherein the sensor captures motions of the athletic performance.

Embodiment 109. The computing system of embodiment 108, wherein the sensor captures at least 30 images per second.

Embodiment 110. The computing system of any one of embodiments 85-109, wherein the digital application further comprises a push alarm and/or push notifications for reminding the subject to complete a digital therapeutic module.

Embodiment 111. The computing system of any one of embodiments 85-110, wherein the digital apparatus comprises:
a digital instruction generation unit configured to generate digital therapeutic modules for enhancing performance of the user, generate digital instructions based on the digital therapeutic modules, and provide the digital instructions to the user; and
an outcome collection unit configured to collect the user's execution outcomes of the digital instructions.

Embodiment 112. The computing system of any one of embodiment 111, wherein the digital instruction generation unit generates the digital therapeutic modules based on the inputs from the healthcare provider.

Embodiment 113. The computing system of any one of embodiments 111-112, wherein the digital instruction generation unit generates the digital therapeutic modules based on information received from the user.

Embodiment 114. The computing system of embodiment 113, wherein the information is received from the user comprises at least one of basal factors, medical information, and digital therapeutics literacy of the user,
the basal factors including the user's activity, heart rate, sleep, and diet,
the medical information including the user's electronic medical record (EMR), family history, genetic vulnerability, and genetic susceptibility, and
the digital therapeutics literacy including the user's accessibility, and technology adoption to the digital therapeutics and the apparatus.

Embodiment 115. The computing system of any one of embodiments 111-114, wherein the outcome collection unit collects the execution outcomes of the digital instructions by monitoring the user's adherence to the digital instructions or allowing the user to directly input the user's adherence to the digital instructions.

Embodiment 116. The computing system of any one of embodiments 111-115, wherein the generation of the digital instructions at the digital instruction generation unit and the collection of the user's execution outcomes of the digital instructions at the outcome collection unit are repeatedly executed several times with multiple feedback loops, and
the digital instruction generation unit generates the user's digital instructions for this cycle based on the user's digital instructions in the previous cycle and the execution outcome data on the user's digital instructions in the previous cycle collected at the outcome collection unit.

Embodiment 117. A non-transitory computer readable medium having stored thereon software instructions for enhancing performance of the user described above that, when executed by a processor, cause the processor to:

display, by an electronic device to the user, the one or more first modules described above, each of the one or more first modules comprising instructions for the user to follow;

sense, by the sensor described above in the electronic device, adherence by the user to the instructions of the one or more first modules;

transmit, by the electronic device, adherence information based on the adherence, to a server;

receive, from the server, one or more second instructions based on the adherence information; and

display, to the user, the one or more second modules described above, the one or more second modules comprising the one or more second instructions.

Embodiment 118. The method of embodiments 21-42, the system of embodiments of 43-84, the computing system of embodiments of 85-116, or the non-transitory computer readable medium of embodiment 117, wherein said one or more first modules comprise a concentration module I for inducing dopamine secretion, an anxiety and/or stress module, a concentration module II for inducing melatonin secretion, and a coordination and agility module.

Embodiment 119. The method of embodiments 21-42, the system of embodiments of 43-84, the computing system of embodiments of 85-116, or the non-transitory computer readable medium of embodiment 117, wherein the adherence is an adherence to the first instructions of the concentration module I, and the adherence information based on the adherence to the first instructions of the concentration module I is used to generate the second instructions only of the concentration module I.

Embodiment 120. The method of embodiments 21-42, the system of embodiments of 43-84, the computing system of embodiments of 85-116, or the non-transitory computer readable medium of embodiment 117, wherein the adherence is an adherence to the first instructions of the anxiety and/or stress module, and the adherence information based on the adherence to the first instructions of the anxiety and/or stress module is used to generate the second instructions only of the anxiety and/or stress module.

Embodiment 121. The method of embodiments 21-42, the system of embodiments of 43-84, the computing system of embodiments of 85-116, or the non-transitory computer readable medium of embodiment 117, wherein the adherence is an adherence to the first instructions of the concentration module II, and the adherence information based on the adherence to the first instructions of the concentration module II is used to generate the second instructions only of the concentration module II.

Embodiment 122. The method of embodiments 21-42, the system of embodiments of 43-84, the computing system of embodiments of 85-116, or the non-transitory computer readable medium of embodiment 117, wherein the adherence is an adherence to the first instructions of the a coordination and agility module, and the adherence information based on the adherence to the first instructions of the a coordination and agility module is used to generate the second instructions only of the a coordination and agility module.

1. A method for use in an apparatus to enhance performance of a user, the method comprising:
generating one or more performance enhancing instructions that cause, based on the user's performance of the one or more performance enhancing instructions, at least one physiological changes in the user's physiological mechanism to cope with at least one of anxiety or stress; and
providing the user the one or more performance enhancing instructions via the apparatus.

2. The method of claim 1, wherein the at least one physiological changes includes at least one of dopamine secretion induction, oxytocin secretion induction, cortisol level reduction, melatonin secretion induction, or synapsing promotion.

3. The method of claim 1, wherein the one or more performance enhancing instructions comprise a first concentration enhancing digital instruction, a second concentration enhancing digital instruction, an anxiety/stress reducing digital instruction, and a coordination & agility enhancing digital instruction.

4. A method of enhancing performance of a user, the method comprising
providing, by an electronic device to the user, one or more first modules selected from the group consisting of a concentration module I for inducing dopamine secretion, an anxiety and/or stress module, a concentration module II for inducing melatonin secretion, and a coordination and agility module, each of the one or more first modules comprising one or more first instructions for the user to follow,
wherein the electronic device (i) comprises a sensor sensing adherence by the user to the first instructions of the one or more first modules, (ii) transmits adherence information based on the adherence, to a server, and (iii) receives one or more second instructions from the server based on the adherence information; and
providing, by the electronic device to the user, one or more second modules selected from the group consisting of a concentration module I for inducing dopamine secretion, an anxiety and/or stress module, a concentration module II for inducing melatonin secretion, and a coordination and agility module, the one or more second modules comprising the one or more second instructions.

5. The method of claim 4, wherein the one or more first modules comprise the concentration module Ito strengthen concentration of the user, and the concentration module I comprises one or more first instructions directed to affirmative thinking, achievement, and accomplishment.

6. The method of claim 4, wherein the server receives the one or more second instructions from an external reviewer.

7. The method of claim 4, wherein the sensor comprises the GPS sensor and the heart rate sensor.

8. The method of claim 4, wherein the user is an athlete, and the performance is athletic performance.

9. The method of claim 4, wherein the user is a baseball player.

10. The method of claim 4, wherein the user is a skier.

11. The method of claim 8, wherein the sensor captures motions of the athletic performance.

12. The method of claim 11, wherein the sensor captures at least 30 images per second.

13. A system for enhancing performance of a user, comprising:
a digital apparatus configured to execute a digital application comprising one or more first modules, for enhancing performance of a user, wherein the digital apparatus comprises a sensor for sensing adherence by the user to a first set of instructions of the one or more first modules;
a healthcare provider portal configured to provide one or more options to a healthcare provider to perform one or more tasks to prescribe performance enhancement of a user based on information received from the digital application; and
an administrative portal configured to provide one or more options to an administrator of the system to perform one or more tasks to manage access to the system by the healthcare provider.

14. A computing system for enhancing performance of a user, comprising:
a display configured to provide, to the user, one or more first modules selected from the group consisting of a concentration module I for inducing dopamine secretion, an anxiety and/or stress module, a concentration module II for inducing melatonin secretion, and a coordination and agility module, each of the one or more first modules comprising one or more first instructions for the user to follow;
a sensor configured to sense adherence by the user to the instructions of the one or more first modules;
a transmitter configured to transmit adherence information based on the adherence, to a server; and
a receiver configured to receive, from the server, one or more second instructions based on the adherence information,
wherein the display is further configured to provide, to the user, one or more second modules selected from the group consisting of a concentration module I for inducing dopamine secretion, an anxiety and/or stress module, a concentration module II for inducing melatonin secretion, and a coordination and agility module, each of the one or more second modules comprising the one or more second instructions.

15. A non-transitory computer readable medium having stored thereon software instructions for enhancing performance of a user that, when executed by a processor, cause the processor to:
display, by an electronic device to the user, one or more first modules selected from the group consisting of a concentration module I for inducing dopamine secretion, an anxiety and/or stress module, a concentration module II for inducing melatonin secretion, and a coordination and agility module, each of the one or more first modules comprising instructions for the user to follow;
sense, by a sensor in the electronic device, adherence by the user to the instructions of the one or more first modules;
transmit, by the electronic device, adherence information based on the adherence, to a server;
receive, from the server, one or more second instructions based on the adherence information; and
display, to the user, one or more second modules selected from the group consisting of a concentration module I for inducing dopamine secretion, an anxiety and/or stress module, a concentration module II for inducing melatonin secretion, and a coordination and agility module, the one or more second modules comprising the one or more second instructions.
